(19) United States
(12) Patent Application Publication
(10) Pub. No.: US 2008/0161279 A1
(43) Pub. Date: Jul. 3, 2008

(54) Methods of Treating Obesity

(76) Inventor: Gerald L. Wisler, Gladstone, NJ (US)

Correspondence Address: GOODWIN PROCTER LLP;PATENT ADMINISTRATOR, EXCHANGE PLACE, BOSTON, MA, 02109-2881, US

(21) Appl. No.: 11/962,383

(22) Filed: Dec. 21, 2007

Related U.S. Application Data
(60) Provisional application No. 60/876,280, filed on Dec. 21, 2006.

(51) Int. Cl. A61K 31/397; A61K 31/4468; A61K 31/437; A61P 3/04

(52) U.S. Cl. 514/210.02; 514/325; 514/292

(57) ABSTRACT

The invention is directed to methods for treating and/or controlling obesity in a patient. The methods involve combination therapies using a microsomal triglyceride transfer protein (MTP) inhibitor (for example, AEGR-733 and implitapide) and a cholesterol absorption inhibitor (CAI) (for example, ezetimibe).

### RELATED APPLICATIONS

This application claims the benefit of U.S. Provisional Patent Application Ser. No. 60/876,280 filed Dec. 21, 2006, the entire disclosure of which is incorporated by reference herein

### FIELD OF THE INVENTION

This invention relates generally to methods of treating and/or controlling obesity in a patient. More particularly, the invention relates to therapies using a microsomal triglyceride transfer protein (MTP) inhibitor in combination with a cholesterol absorption inhibitor (CAI).

### BACKGROUND

Obesity is a major public health concern and is now recognized as a chronic disease that requires treatment to reduce its associated health risks. It is understood that more than 100 million adults in the United States are overweight or obese. The medical problems caused by overweight and obesity can be serious and often life-threatening, and include diabetes, shortness of breath, gallbladder disease, hypertension, elevated blood cholesterol levels, cancer, arthritis, other orthopedic problems, reflux esophagitis (heartburn), snoring, sleep apnea, menstrual irregularities, infertility and heart trouble. Moreover, obesity and overweight substantially increase the risk of morbidity from hypertension, dyslipidemia, type 2 diabetes, coronary heart disease, stroke, gallbladder disease, osteoarthritis and endometrial, breast, prostate, and colon cancers. Higher body weights are also associated with increases in all-cause mortality. Most or all of these problems are alleviated or improved by permanent significant weight loss. Longevity is likewise significantly increased by permanent significant weight loss. Hence, it is believed that a 2-10% intentional reduction in body weight may reduce morbidity and mortality. There is a clear on-going need for methods for treating obesity that effectively reduce body mass in a patient in need thereof.

Microsomal triglyceride transfer protein (MTP) inhibitors have been developed as potent inhibitors of MTP-mediated neutral lipid transfer activity. MTP catalyzes the transport of triglyceride, cholesteryl ester, and phosphatidylcholine between small unilamellar vesicles.

Cholesterol absorption inhibitors such as ezetimbe impair the intestinal reabsorption of both dietary and hepatically-excreted biliary cholesterol. Ezetimbe, for example, is used for reducing low density lipoprotein cholesterol in patients. Cholesterol absorption inhibitors are not known to be effective, when used in monotherapy, for use in treating obesity or for use as a weight loss agent.

### SUMMARY OF THE INVENTION

The invention provides methods for treating and/or controlling obesity. The method includes administering an MTP inhibitor, such as AEGR-733 or implitapide, in combination with a cholesterol absorption inhibitor (CAI), such as ezetimibe. The MTP inhibitors can be administered at certain lower dosages that are still therapeutically effective when combined with a CAI but yet create fewer or reduced adverse effects when compared to therapies using therapeutically effective dosages of the MTP inhibitors during monotherapy. The administration of one or more MTP inhibitors, when administered in combination with one or more CAIs, may provide an additive or synergistic therapeutic effect, e.g. may result in patient weight loss that is greater than the sum of the expected weight loss due to administration of a MTP inhibitor and CAI when administered alone. In some embodiments, disclosed methods can result in fewer incidences of gastrointestinal adverse events in a patient as compared to administration of a MTP inhibitor alone.

An exemplary method includes a method of treating obesity comprising administering to a patient in need thereof a MTP inhibitor in combination with a cholesterol absorption inhibitor, wherein the administration of the combination results in a greater reduction in body mass of the patient after 12 weeks of daily administration as compared to 12 weeks of daily administration of a cholesterol absorption inhibitor or a MTP inhibitor alone.

For example, a method of treating obesity is disclosed that comprises administering to a patient in need thereof a MTP inhibitor in combination with a cholesterol absorption inhibitor, wherein the administration of the combination results in a greater reduction in body mass of the patient after 12 weeks of daily administration as compared to 12 weeks of daily administration of a cholesterol absorption inhibitor or a MTP inhibitor alone, and wherein the method results in fewer incidences of gastrointestinal adverse events in the patient as compared to administration of a MTP inhibitor alone.

Another exemplary method contemplated by this disclosure includes a method of inducing weight loss in a patient comprising administering to the patient an MTP inhibitor in combination with a cholesterol absorption inhibitor so as to induce weight loss in the patient. In some embodiments, the weight loss achieved, after e.g. 4 weeks, 8 weeks, 12 weeks, or even 6 months, is greater than that achieved by administering the cholesterol inhibitor alone or the MTP inhibitor alone. In an embodiment, weight loss achieved by the disclosed methods is greater than the additive effect of administering the MTP inhibitor alone and the cholesterol absorption inhibitor alone.

### BRIEF DESCRIPTION OF THE DRAWINGS

Throughout this entire disclosure, including the figures and claims, the terms “AEGR-733” and “BMS-201038” have the same meaning and are used interchangeably.

FIG. 1 depicts body mass reduction at 4 weeks, 8 weeks, and 12 weeks of daily administration of AEGR-733 and ezetimibe in the patient study described in Example 1.

FIG. 2 depicts the occurrence rate of gastrointestinal adverse events and the GSRS results of patients assessed at 12 weeks in the patient study described in Example 1.

FIG. 3 depicts body mass reduction at 4 weeks, 8 weeks, and 12 weeks of daily administration of AEGR-733 and ezetimibe for those patients with an initial BMI greater than 30 kg/m² in the patient study as described in Example 1.

FIG. 4 depicts body mass reduction at 4 weeks, 8 weeks, and 12 weeks of daily administration of AEGR-733 and ezetimibe for those patients with an initial BMI less than or equal to 30 kg/m in the patient study as described in Example 1.

### DETAILED DESCRIPTION

The invention relates, in part, to methods of treating and/or controlling obesity comprising administering to a patient in need thereof a MTP inhibitor in combination with a cholesterol absorption inhibitor. Such a patient may have, for example, a body mass index greater than or equal to about 30 kg/m², e.g. between about 30 kg/m and about 60 kg/m before treatment. Alternatively, a patient may have a body mass index between about 25 kg/m and about 30 kg/m² before treatment.

The methods described herein result in a greater reduction in body mass of a patient after, for example, four, eight and/or twelve weeks of daily administration, or 4, 5, and/or 6 months or 1 year of substantially daily administration, as compared to daily administration of a cholesterol absorption inhibitor or a MTP inhibitor alone for the same time interval.

Administering combinations of a MTP inhibitor and a cholesterol absorption inhibitor, under certain circumstances, provide an additive and/or synergistic therapeutic effect, e.g. provide a total reduction in body mass that is greater than the sum of the reduction in body mass resulting from administering a MTP inhibitor or a cholesterol absorption inhibitor alone.

### 1. DEFINITIONS

For convenience, certain terms used in the specification, examples, and appended claims are collected in this section.

The phrase “combination therapy,” as used herein, refers to co-administering an MTP inhibitor, for example, AEGR-733 and implitapide, or a combination thereof, and CAI, for example, ezetimibe, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules or tablets for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy can also embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination may be administered to a patient simultaneously or sequentially. It will be appreciated that the components may be present in the same pharmaceutically acceptable carrier and, therefore, are administered simultaneously. Alternatively, the active ingredients may be present in separate pharmaceutical carriers, such as, conventional oral dosage forms, that can be administered either simultaneously or sequentially.

The terms, “individual,” “patient,” or “subject” are used interchangeably herein and include any mammal, including animals, for example, primates, for example, humans, and other animals, for example, dogs, cats, swine, cattle, sheep, and horses. The compounds of the invention can be administered to a mammal, such as a human, but can also be other mammals, for example, an animal in need of veterinary treatment, for example, domestic animals (for example, dogs, cats, and the like), farm animals (for example, cows, sheep, pigs, horses, and the like) and laboratory animals (for example, rats, mice, guinea pigs, and the like).

The phrase “minimizing adverse effects,” “reducing adverse events,” or “reduced adverse events,” as used herein refer to an amelioration or elimination of one or more undesired side effects associated with the use of MTP inhibitors of the present invention. Side effects of traditional use of the MTP inhibitors include, without limitation, diarrhea, nausea, gastrointestional disorders, steatorrhea, abdominal cramping, distention, elevated liver function tests, fatty liver (hepatic steatosis); hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). Accordingly, the methods described herein provide an effective therapy while at the same time may cause fewer or less significant adverse events as compared to larger monotherapies alone.

In certain embodiments, side effects are partially eliminated. As used herein, the phrase “partially eliminated” refers to a reduction in the severity, extent, or duration of the particular side effect by at least 30%, 40%, 50%, 60%, 70%, 80%, 90%, 95% and 99% relative to that found by administering 25 mg/day of AEGR-733 during monotherapy or either 80 mg/day or 160 mg/day of implitapide during monotherapy. In certain embodiments, side effects are completely eliminated. Those skilled in the art are credited with the ability to detect and grade the severity, extent, or duration of side effects as well as the degree of amelioration of a side effect. Assessment of side effects can be conducted using assessments and/or tests as known to those skilled in the art. For example, gastrointestinal side effects can be assessed, for example, using the Gastrointestinal Symptom Rating Scale. In some embodiments, two or more side effects are ameliorated.

The Gastrointestinal Symptom Rating Scale (“GSRS”) is an assessment tool for patients with general gastrointestinal complaints, and has been extensively validated in previous studies. The GSRS includes up to 15 items that addresses different gastrointestinal symptoms and typically uses a 7-point Likert response scale with verbal descriptors. The response scale is designed to measure the amount of discomfort a patient has experienced (none at all, minor, mild, moderate, moderately severe, severe, and very severe). A higher score in a GSRS cluster indicates more discomfort, with the scale from 1 (no discomfort) to 7. The recall period can refer, for example, to the past week. The 15 exemplary items can combine into five symptom clusters labeled reflux, abdominal pain, indigestion, diarrhea, and constipation. From individual items within a cluster, a mean score is calculated.

The term “synergistic” refers to two or more agents, e.g. a MTP inhibitor and a CAI, when taken together, produce a total joint effect that is greater than the sum of the effects of each drug when taken alone.

The term, “therapeutically effective” refers to the ability of an active ingredient, alone or in combination with another active agent, to elicit the biological or medical response that is being sought by a researcher, veterinarian, medical doctor or other clinician.

The term, “therapeutically effective amount” includes the amount of an active ingredient, or combination of active ingredients, that will elicit the biological or medical response that is being sought by the researcher, veterinarian, medical doctor or other clinician. The compounds of the invention are administered in amounts effective for treating and/or reducing obesity. Alternatively, a therapeutically effective amount of an active ingredient is the quantity of the compound required to achieve a desired therapeutic and/or prophylactic effect, such as the amount of the active ingredient that results in the prevention of or a decrease in the symptoms associated with the condition (for example, to meet an end-point).

The terms, “pharmaceutically acceptable” or “pharmacologically acceptable” refer to molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Pharmaceutically acceptable salts of the disclosed compounds can be synthesized, for example, from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. The terms “racemate,” “racemic mixture” or “racemic modification” refer to a mixture of equal parts of enantiomers.

### 2. METHODS OF THE INVENTION

In general the invention provides methods for treating and/or controlling obesity using one or more MTP inhibitors, for example, AEGR-733 or implitapide, in combination with a cholesterol absorption inhibitor, for example ezetemibe. The MTP inhibitors can be used at dosages lower than those already found to result in one or more adverse events, for example, gastrointestinal disorders, abnormalities in liver functional and/or hepatic steatosis (for example, 25 mg/day of AEGR-733, 80 mg/day of implitapide and 160 mg/day of implitapide have been found to cause gastrointestinal disorders, abnormalities in liver function and/or hepatic steatosis) but at doses which are therapeutically effective when combined with a cholesterol absorption inhibitor, for example, ezetimibe. The dosages need not be smaller but may additionally and/or optionally be administered less frequently. It is contemplated that such a combination may be effective at treating and/or controlling obesity, e.g. effective in promoting weight reduction, in a patient even when larger dosages of AEGR-733 are administered together with a dose of a cholesterol absorption inhibitor.

Also contemplated herein are methods of treating obesity-related disorders such as those associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease or syndrome, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure, e.g, children with acute lymphoblastic leukemia. Further examples of obesity-related disorders are Metabolic Syndrome, also known as Syndrome X, insulin resistance syndrome, sexual and reproductive dysfunction, such as infertility, hypogonadism in males and hirsutism in females, acanthosis nigricans, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compositions of the present invention also are useful for reducing the risk of secondary outcomes of obesity, such as reducing the risk of left ventricular hypertrophy. Methods for treating patients at risk of obesity, such as those patients who are overweight, e.g. with a BMI of between about 25 and 30 kg/m², are also contemplated. Therefore, the present invention includes a method of treating each of the foregoing diseases or conditions in a patient with one or more of the diseases or conditions comprising administering to the patient in need of such treatment dosage combinations of a MTP inhibitor compound and cholesterol absorption inhibitor.

Also provided herein is a method of inducing weight loss in a patient comprising administering to the patient an MTP inhibitor in combination with a cholesterol absorption inhibitor so as to induce weight loss in the patient. Such weight loss may be greater than that achieved by administering the cholesterol inhibitor alone or the MTP inhibitor alone, for example, the weight loss may be greater than the additive effect of administering the MTP inhibitor alone and the cholesterol absorption inhibitor alone.

“Obesity” is a condition in which there is an excess of body fat. Typically, the definition of obesity is based on the Body Mass Index (BMI), which is calculated as body weight per height in meters squared (kg/m²). Obesity refers to a condition whereby an otherwise healthy patient has a Body Mass Index (BMI) greater than or equal to 30 kg/m² or a condition whereby a patient with at least one co-morbidity has a BMI greater than or equal to 27 kg/m². Obesity can also refer to those patients with a waist-to-hip ratio of 0.85 or more for women and 1.0 or more for men. Obesity can also refer to patients with a waist circumference of about 102 cm for males and about 88 cm for females.

A patient at risk of obesity is an otherwise healthy subject with a BMI of 25 kg/m² to less than 30 kg/m² or a subject with at least one co-morbidity with a BMI of 25 kg/m² to less than 27 kg/m². Alternatively or additionally, a patient at risk of obesity can refer to those patients with a waist-to-hip ratio of, e.g. 0.8 to 0.9 (women) and 0.9 to 1.0 (men). Such a patient may be in need of controlling obesity.

The increased risks associated with obesity occur at a lower Body Mass Index (BMI) in Asian patients or patients with Asian ancestry. In Asian countries, including Japan, obesity may refer to a condition whereby a patient with at least one obesity-induced or obesity-related co-morbidity, that requires weight reduction or that would be improved by weight reduction, has a BMI greater than or equal to 25 kg/m². For Asian patients a subject at risk of obesity is a subject with a BMI of greater than 23 kg/m² and less than 25 kg/m².

(a) Combination Therapies Using MTP inhibitors and Cholesterol Absorption Inhibitors

The method comprises a combination therapy, which can be achieved by co-administering to the mammal a MTP inhibitor and a cholesterol absorption inhibitor. The MTP inhibitor and the cholesterol absorption inhibitor can be administered as a (i) single dosage form or composition, (ii) simultaneously as separate dosage forms or pharmaceutical compositions, (iii) sequentially, as separate dosage forms starting with the MTP inhibitor and then administering the cholesterol absorption inhibitor, or starting with the cholesterol absorption inhibitor and then administering the MTP inhibitor, (iv) successively, separated by for example 1-4 hours, 1-8 hours or 1-12 hours, a day, or 2 or more days, e.g. 2 to 3 days, or (v) individually followed by the combination. The methods disclosed herein may occur before, during, or after other dosing regimens that may include, for example MTP inhibitors, cholesterol absorption inhibitors, other agents for treating obesity, and/or agents for reducing cholesterol such as for example a HMG-CoA reductase inhibitor, a bile acid sequestrant, a fibric acid derivative, niacin, squalene synthetase inhibitors, ACAT inhibitors, and/or CETP inhibitors.

In some embodiments, the MTP inhibitor is administered in escalating doses. Such escalating doses may comprise a first dose level and a second dose level. In other embodiments, escalating doses may comprise at least a first dosage level, a second dosage level, and a third dosage level, and optionally a fourth, fifth, or sixth dosage level. The cholesterol absorption inhibitor may be provided in one dosage level when in administered in combination with a MTP inhibitor, or may be administered in escalating doses.

A first, second, third or more dosage levels can be administered to a patient for about 2 days to about 6 months or more in duration. For example, first, second and/or third dose levels are each administered to a subject for about 1 week to about 26 weeks, or about 1 week to about 12 weeks, or about 1 week to about four weeks. Alternatively, the first, second and/or third dosage levels are administered to a subject for about 2 days to about 40 days or to about 6 months.

The methods disclosed herein may reduce the body mass of a patient due to a decrease in caloric fat absorption. For example, after twelve weeks of a disclosed therapy, a patient may have a 2%, 3% or more reduction in body mass. For a patient with a BMI of greater than 30 kg/m², such a patient may have 3%, 3.5%, 5%, 6%, 7%, 8%, 9%, 10% or more reduction in body mass after, for example, one, two, four, eight, twelve, twenty-four, or more weeks of a disclosed therapy.

The MTP inhibitor and/or the cholesterol absorption inhibitor each may be administered in a therapeutically effective amount and/or each in a synergistically effective amount. Such dosages of a MTP inhibitor and/or a cholesterol absorption inhibitor may, while not effective when used in monotherapy, may be effective when used in the combinations disclosed herein.

Administration of the MTP inhibitor and the cholesterol absorption inhibitor may result in fewer gastrointestinal adverse events, such as GI disorders, as compared to administration of a MTP inhibitor alone. In some embodiments, administration of the MTP inhibitor and the cholesterol absorption inhibitor may result in greater weight loss and fewer gastrointestinal adverse events as compared to administration of a MTP inhibitor or cholesterol absorption inhibitor alone.

MTP Inhibitors

In one embodiment, the MTP inhibitor may be AEGR-733. As used herein, the phrase “BMS-201038” or “AEGR-733” refers to a compound known as N-(2,2,2-Trifluorethyl)-9-[4-[4-[[[4′-(trifluoromethyl)[1,1′biphenyl]-2-Yl]carbonyl]amino]-1-piperidinyl]butyl]9H-fluorene-9-carboxamide, having the formula:

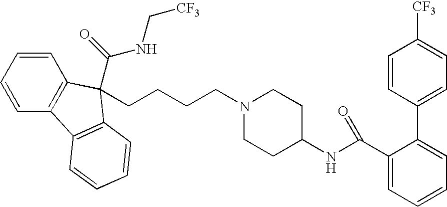

the piperidine N-oxide thereof, and stereoisomers, and pharmaceutically acceptable salts and esters thereof.

In another embodiment, the MTP inhibitor may include benzimidazole-based analogues of AEGR-733, for example, a compound having the formula shown below:

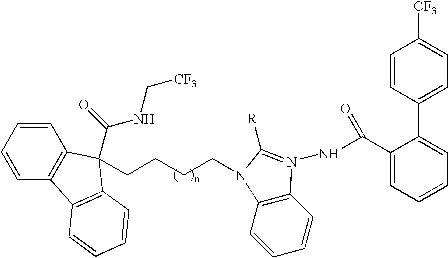

where n can be 0 to 10, and stereoisomers thereof, and pharmaceutically acceptable salts and esters thereof.

In another embodiment, the MTP inhibitor may be implitapide. As used herein, the phrase “implitapide” refers to a compound (2S)-2-cyclopentyl-2-[4-[(2,4-dimethyl-9H-pyrido[2,3-b]indol-9-yl)methyl]phenyl]-N-[(1S)-2-hydroxy-1-phenylethyl]ethanamide, and having the structure shown below:

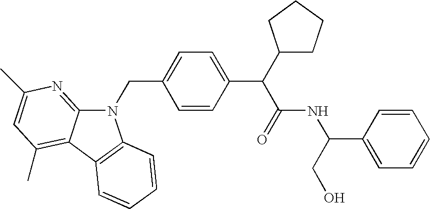

and stereoisomers thereof, and pharmaceutically acceptable salts and esters thereof.

In another embodiment, the MTP inhibitor may be JTT-130m including pharmaceutically acceptable salts and esters thereof, described in Aggarwal, et al., BMC CARDIOVASC. DISORD. 27; 5(1):30 (2005). In another embodiment, the MTP inhibitor may be CP-346086 including pharmaceutically salts and esters thereof, described in Chandler, et al., J. LIPID. RES. 44(10):1887-901 (2003).

Other MTP inhibitors include those developed by Surface Logix, Inc. e.g., SLx-4090.

Cholesterol Absorption Inhibitors

In one embodiment, the CAI may be ezetimibe (also known as Zetia). As used herein, the phrase “ezetimibe” refers to a compound having the structure shown below:

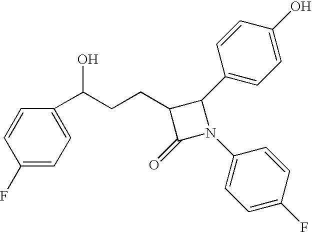

and stereoisomers thereof, and pharmaceutically acceptable salts and esters thereof.

In one embodiment, the CAI may be MD-0727 including pharmaceutically acceptable salts and esters thereof. In another embodiment, the CAI may be FM-VP4. As used herein, the phrase “FM-VP4” refers to a compound the structure of which is set forth below:

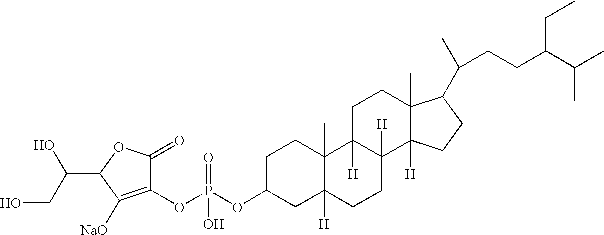

and stereoisomers thereof, and pharmaceutically acceptable salts and esters thereof.

In another embodiment, the CAI may be the structure below, as described in Ritter et al., Org. Biomol. Chem., 3(19), 3514-3523, (2005):

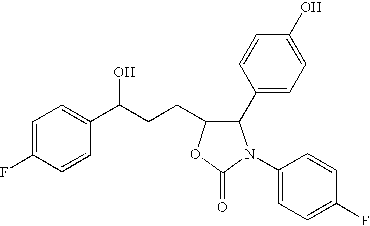

and stereoisomers thereof, and pharmaceutically acceptable salts and esters thereof.

In another embodiment, the CAI may be LPD179. As used herein, the phrase “LDP179” refers to a compound having the structure set forth below:

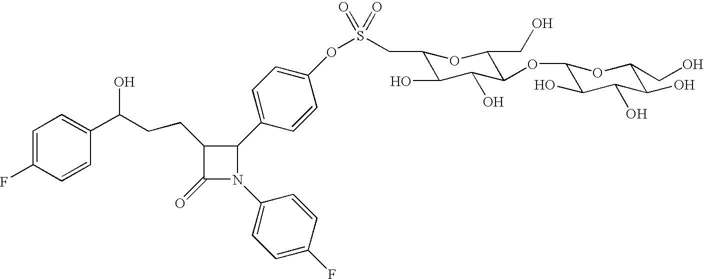

and stereoisomers thereof, and pharmaceutically acceptable salts and esters thereof.

In another embodiment, the CAI may be LPD84. As used herein, the phrase “LPD84” refers to a compound having the structure set forth below:

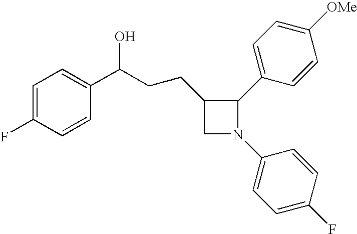

and stereoisomers thereof, and pharmaceutically acceptable salts and esters thereof.

In another embodiment, the CAI may be LPD145. As used herein, the phrase “LPD145” refers to a compound having the structure set forth below:

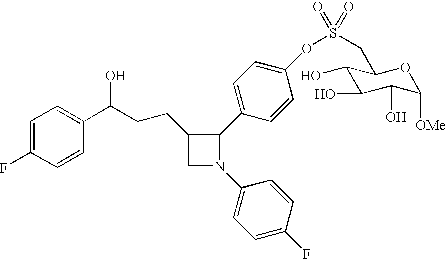

and stereoisomers thereof, and pharmaceutically acceptable salts and esters thereof.

Other cholesterol absorption inhibitors include the CAI identified as AVE5530.

Other useful exemplary ezetimibe derivatives, their synthesis and use are described, for example, in International Application Publication No. WO 2005/033100.

For example, a MTP inhibitor can be administered in combination with ezetimibe. Ezetimibe may be co-administered at a dosage in the range of 0.01 to 100 mg/day, more preferably at a dosage in the range of 1 to 50 mg/day, or 1 to 25 mg/day, for example, administered at a dosage of 5 mg/day, 10 mg/day, 15 mg/day, 20 mg/day, or 25 mg/day. In an embodiment, ezetimibe may be administered at a dosage of 10 mg/day.

(b) Therapies Using AEGR-733 and Ezetimibe

In one aspect, the invention provides a method of treating and/or controlling obesity comprising administering a combination of ezetimibe and AEGR-733 to a patient daily.

Exemplary dosages for administration of AEGR-733 in combination with a cholesterol absorption inhibitor, e.g. ezetimibe, include a dosage of about 1 mg/day to about 25 mg/day, e.g. 2.5 mg/day, 5 mg/day, 7.5 mg/day, 10 mg/day, 15 mg/day or 20 mg/day of AEGR-733. In some embodiments, doses of about 10-100 mg/day, 20-80 mg/day, can be administered, for example, a dosage of 20 mg/day, 30 mg/day, 40 mg/day, 60 mg/day or 80 mg/day.

In an exemplary dose escalation regimen, the first dose level of AEGR-733 may be from about 2 to about 13 mg/day, and/or the second dose level may be about 5 to about 30 mg/day.

In an exemplary protocol, AEGR-733 initially is administered at a first dosage in the range of 2.5 to 7.5 mg/day for at least 4 weeks, is then administered at a second dosage in the range of 5 to 10 mg/day for at least 4 weeks, and is then administered at a third dosage in the range of 7.5 to 12.5 mg/day for at least 4 weeks. Such dosage regimens may each be in combination with, e.g., 10 mg/day of ezetimibe.

The first dosage of AEGR-733 can be for example 2.5 mg/day or 5 mg/day for about 4 weeks. The second dosage of AEGR-733 can be 7.5 mg/day for about 4 weeks. The third dosage of AEGR-733 can be 10 mg/day. In certain embodiments, the second dosage is administered immediately following the first dosage, i.e., the second dosage is administered starting at five weeks from the initial first dosage. Similarly, in certain other embodiments, the third dosage of AEGR-733 is administered immediately following the second dosage, e.g., the second dosage is administered at nine weeks from the initial first dosage.

Optionally, the method may include administering a second, third, or fourth dosage period of AEGR-733 alone, or in combination with ezetimibe. Such a fourth dosage may be in the range of 7.5-12.5 mg/day of AEGR-733 or more. A fourth dosage period may occur immediately after the second or third dosage, or may occur after a time interval, for example, a day, days, a week, or weeks after the third dosage. The fourth dosage may be administered to the subject for 1, 2, 3, 4 or more weeks.

(c) Therapies Using Implitapide and Ezetimibe

In one aspect, the invention provides a method of treating and/or controlling obesity comprising administering a combination of ezetimibe and implitapide to a patient daily.

Implitapide may be administered at a dosage in the range of 0.01 to 60 mg/day, more preferably in the range of 20 to 60 mg/day, for example, 20 mg/day, 25 mg/day, 30 mg/day, 35 mg/day, 40 mg/day or 60 mg/day. Ezetimibe can be coadministered with implitipide at a dose of about 10 mg/day.

### 3. FORMULATION AND ADMINISTRATION OF THE ACTIVE INGREDIENTS

In certain embodiments, the MTP inhibitor (for example, AEGR-733 and implitapide) and the CAI (for example, ezetimibe) are administrated orally. For oral administration, the active ingredients may take the form of solid dose forms, for example, tablets (both swallowable and chewable forms), capsules or gelcaps, prepared by conventional means with pharmaceutically acceptable excipients and carriers such as binding agents (e.g. pregelatinised maize starch, polyvinylpyrrolidone, hydroxypropylmethylcellulose and the like), fillers (e.g. lactose, microcrystalline cellulose, calcium phosphate and the like), lubricants (e.g. magnesium stearate, talc, silica and the like), disintegrating agents (e.g. potato starch, sodium starch glycollate and the like), wetting agents (e.g. sodium laurylsulphate) and the like. Such tablets may also be coated by methods well known in the art.

Although less preferred, it is contemplated that the active ingredients may be formulated for, and administered by, non-parental routes, for example, by intravenous routes, intramuscular routes, and by absorption through mucous membranes. It is contemplated that such formulations and non-parenteral modes of administration are known in the art.

The dosages described above may be administered in single or divided dosages of one to four times daily. The MTP inhibitor and CAI may be employed together in the same dosage form or in separate dosage forms taken at the same time, or at different times.

In certain embodiments, the methods disclosed herein may minimize at least one of side effects associated with the administration of AEGR-733 and/or implitapide and/or ezetimibe alone. Such side effects include, for example, diarrhea, nausea, gastrointestinal disorders, steatorrhea, abdominal cramping, distention, elevated liver function tests such as increases in liver enzymes such as alanine, minor fatty liver; hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). In some embodiments the minimization of the side effect is determined by assessing the grade, severity, extent, or duration by subject questionnaire.

### EXAMPLES

The examples that follow are intended in no way to limit the scope of this invention but are provided to illustrate the methods present invention. Many other embodiments of this invention will be apparent to one skilled in the art.

### Example 1

### AEGR-733/Ezetimibe Combination Therapy

This study is designed to show that doses of AEGR-733 in combination with ezetimibe, can provide clinically significant reductions in body mass. The primary parameter of efficacy in this study is the percentage change in body mass after 12 weeks of therapy.

Approximately 85 subjects were randomized into one of three treatment arms with equal probability. The subjects had a baseline LDL of 130-250 mg/dL and baseline triglyceride level of less than 400 mg/dL. Enrolled patients had initial BMIs of both greater than and less than 30 kg/m². Patients were not instructed to implement lifestyle changes to induce weight loss. Patients were placed on a heart healthy low fat diet 4-5 weeks prior to start of dosing.

In treatment arm 1, subjects received two capsules: AEGR-733 (5 mg) plus an ezetimibe placebo. In effect, treatment arm 1 represents monotherapy with AEGR-733. In treatment arm 2, subjects received two capsules: an AEGR-733 placebo plus ezetimibe (10 mg). In effect treatment arm 2 represents monotherapy with ezetimibe. In treatment arm 3, subjects received two capsules: an AEGR-733 (5 mg) capsule plus an ezetimibe (10 mg) capsule. Treatment arm 3 patients, in effect, received a combination therapy.

After 4 weeks of treatment, subjects in arms 1 and 3 received a step-up in concentration of AEGR-733 from 5 mg to 7.5 mg for 4 weeks. Thereafter, subjects in arms 1 and 3 received a second step-up in concentration in AEGR-733 from 7.5 mg to 10 mg for 4 more additional weeks of treatment. Subjects in arm 2 continued to receive AEGR-733 matching placebo for the entire 12 weeks of treatment. Subjects randomized to ezetimibe 10 mg in arms 2 and 3 and ezetimibe placebo in arm 1 remained on these doses for the entire 12-week treatment period.

Throughout the study, changes in body weight of the subjects are measured as part of vital signs collection.

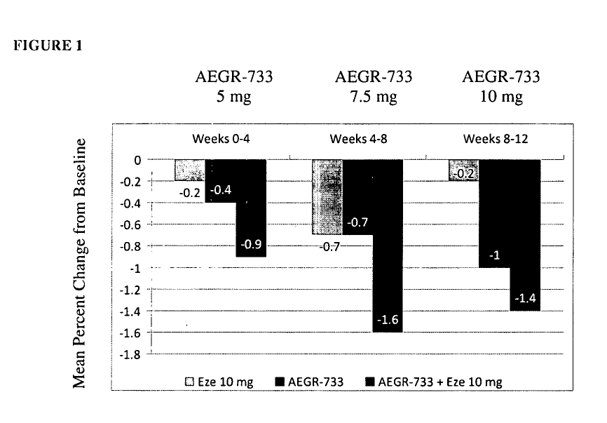

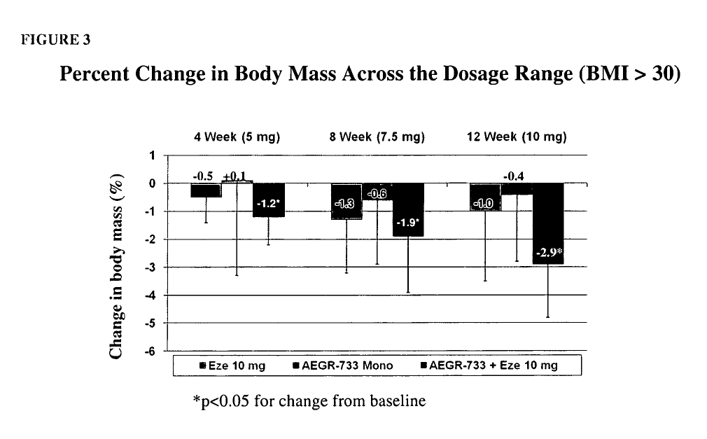

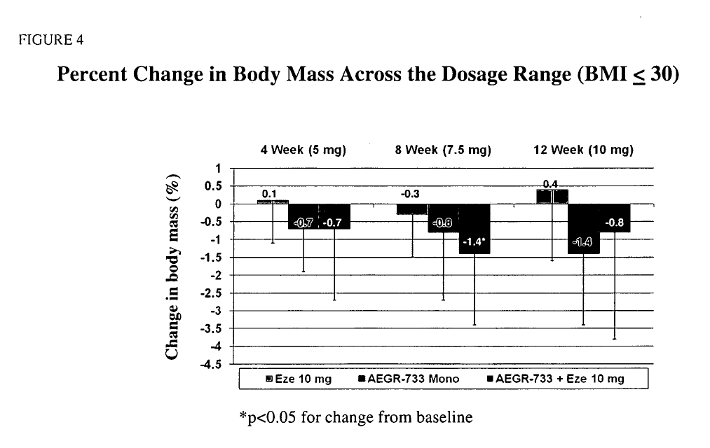

FIG. 1 shows change in body mass of patients of each arm at weeks 4, 8, and 12 respectively. FIG. 3 depicts the same data only for those patients with a BMI of greater than 30 kg/m². FIG. 4 depicts the same data only for those patients with a BMI of less than or equal to 30 kg/m².

FIG. 1 depicts graphically the percent change in body mass for patients in arm 3 was about −1.4% after 12 weeks of the trial as compared to those patients administered ezetimibe alone (−0.2%) or AEGR-733 alone (−1.0%). Patients in arm 3 therefore had a greater percent change in body mass as compared to any expected additive effect for patients administered ezetimibe or AEGR-733 alone, e.g. −1.4% vs. (−1.0%+−0.2%=−1.2%). In this analysis of the entire cohort, changes for patients, at weeks 4, 8, and 12 in the combination group had statistically significant change from baseline weight, p<0.05.

Similarly, patient groups with an initial BMI less than 30 mg/k² (FIG. 4) in arm 3 after 12 weeks had a −0.8% percent change in body mass as compared to patients administered ezetimibe (+0.4%) or AEGR-733 (−1.4%) alone; patient groups with an initial BMI greater than 30 mg/k² (FIG. 3) in arm 3 after 12 weeks had a −2.9% percent change in body mass as compared to patients administered ezetimibe (−1.0%) or AEGR-733 (−0.4%) alone.

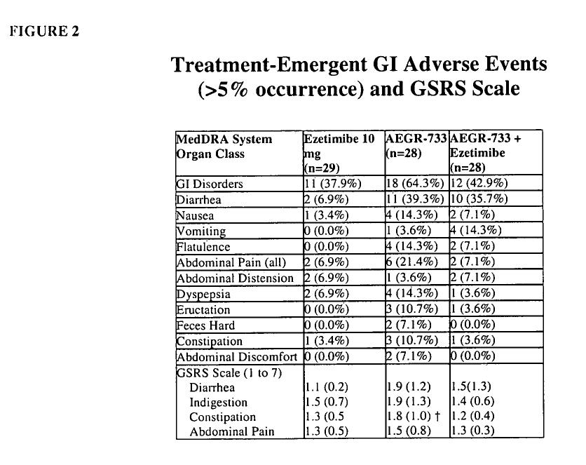

Patients were assessed for gastrointestinal adverse events and GSRS was taken at baseline (before the trial began), and after 4, 8, and 12 weeks of trial as described above. FIG. 2 depicts the occurrence rate of gastrointestinal adverse events and the GSRS results of patients when assessed at 12 weeks.

### EQUIVALENTS

It is understood that the disclosed invention is not limited to the particular methodology, protocols, and dosages described as these may vary. It is also to be understood that the terminology used herein is for the purpose of describing particular embodiments only, and is not intended to limit the scope of the present invention which will be limited only by the appended claims.

### INCORPORATION BY REFERENCE

The entire disclosure of each of the patent documents and scientific articles referred to herein is incorporated by reference for all purposes.

### Patents/Patent Applications

WO 2005/087234, U.S. Ser. No. 11/582,835, PCT/US06/40953

What is claimed is:

1. A method of treating and/or controlling obesity comprising administering to a patient in need thereof a MTP inhibitor in combination with a cholesterol absorption inhibitor.

2. The method of claim 1, wherein the patient is a human.

3. The method of claim 1 or 2, wherein the patient has a body mass index greater than or equal to about 30 kg/m² before the administration.

4. The method of claim 1 or 2, wherein the patient has a body mass index between about 30 kg/m² and about 60 kg/m² before the administration.

5. The method of claim 1, wherein the patient has a body mass index of between about 25 kg/m² and about 30 kg/m² before the administration.

6. The method of any one of claims 1-5, wherein the MTP inhibitor and the cholesterol absorption inhibitor are administered sequentially.

7. The method of any one of claims 1-5, wherein the MTP inhibitor and the cholesterol absorption inhibitor are administered substantially simultaneously.

8. The method of claims 6 or 7, wherein the MTP inhibitor and the cholesterol absorption inhibitor are administered in separate dosage forms.

9. The method of claim 7, wherein the MTP inhibitor and the cholesterol absorption inhibitor are administered as a single dosage form.

10. The method of any one of claims 1-9, wherein administering the MTP inhibitor in combination with a cholesterol absorption inhibitor provides a synergistic therapeutic effect.

11. The method of claim 10, wherein the MTP inhibitor is administered in a synergistically effective amount.

12. The method of claim 10 or 11, wherein the cholesterol absorption inhibitor is administered in a synergistically effective amount.

13. The method of any one of claims 1-12, wherein the cholesterol absorption inhibitor is selected from the group consisting of: ezetimibe, MD-0727, FM-VP4, LPD84, LPD179, LPD 145 and AVE5530.

14. The method of any one of claims 1-13, wherein the cholesterol absorption inhibitor is ezetimibe.

15. The method of claim 14, wherein ezetimibe is administered at a dosage of about 1 to about 50 mg/day.

16. The method of claim 15, wherein ezetimibe is administered at a dosage of about 10 mg/day.

17. The method of any one of claims 1-16, wherein the MTP inhibitor is selected from at least one of: AEGR-733, implitapide, JTT-130, CP-346086, and SLx-4090.

18. The method of 17, wherein the MTP inhibitor is selected from at least one of: implitapide and AEGR-733.

19. The method of claim 18, wherein the MTP inhibitor is AEGR-733.

20. The method of claim 19, wherein AEGR-733 is administered at a dosage between about 1 to about 25 mg/day inclusive.

21. The method of claim 19, wherein AEGR-733 is administered at a dosage between about 20 mg/day and about 80 mg/day.

22. The method of claim 19, wherein AEGR-733 is administered at about 1 mg/day to about 12 mg/day.

23. The method of claim 22, wherein AEGR-733 is administered daily for at least twelve weeks.

24. The method of claim 22, wherein AEGR-733 is administered at 5 mg/day for at least four weeks.

25. The method of claim 19, comprising administering AEGR-733 at a first dosage of about 1 to about 7.5 mg/day for a first interval of at least four weeks.

26. The method of claim 25, wherein the first dosage is about 5 mg/day.

27. The method of claim 25, further comprising administering AEGR-733 at a second dosage of about 5 mg/day to about 12 mg/day for a second interval of at least four weeks.

28. The method of claim 27, wherein the second dosage is about 7.5 mg/day.

29. The method of claim 27 or 28, further comprising administering AEGR-733 at a third dosage of about 6 mg to about 15 mg/day for a third interval of at least four weeks.

30. The method of claim 29, wherein the third dosage is 10 mg/day.

31. The method of claim 18, wherein the MTP inhibitor is implitapide.

32. The method of claim 31, wherein implitapide is administered at a dosage between about 0.01 to about 60 mg/day inclusive.

33. The method of claim 32, wherein implitapide is administered at about 20 mg/day to about 40 mg/day.

34. The method of claim 33, wherein implitapide is administered daily for at least twelve weeks.

35. The method of claim 31, wherein implitapide is administered at about 20 mg/day to about 40 mg/day for at least four weeks.

36. The method of any one of claims 1-35, wherein the incidence of gastrointestinal adverse events in the patient is less than 5%.

37. The method of any one of claims 1-35, wherein the method results in fewer incidences gastrointestinal adverse events in the patient as compared to administration of a MTP inhibitor alone.

38. The method of any one of claims 1-35, further comprising assessing a patient using a GSRS scale.

39. The method of claim 38, wherein the reported GSRS is between 1 and 2.

40. A method of treating obesity comprising administering to a patient in need thereof a MTP inhibitor in combination with a cholesterol absorption inhibitor, wherein the administration of the combination results in a greater reduction in body mass of the patient after 12 weeks of daily administration as compared to 12 weeks of daily administration of a cholesterol absorption inhibitor or a MTP inhibitor alone.

41. A method of treating obesity comprising administering to a patient in need thereof a MTP inhibitor in combination with a cholesterol absorption inhibitor, wherein the administration of the combination results in a greater reduction in body mass of the patient after 12 weeks of daily administration as compared to 12 weeks of daily administration of a cholesterol absorption inhibitor or a MTP inhibitor alone, and wherein the method results in fewer incidences of gastrointestinal adverse events in the patient as compared to administration of a MTP inhibitor alone.

42. A method of inducing weight loss in a patient comprising administering to the patient an MTP inhibitor in combination with a cholesterol absorption inhibitor so as to induce weight loss in the patient.

43. The method of claim 42, wherein the weight loss is greater than that achieved by administering the cholesterol inhibitor alone or the MTP inhibitor alone.

44. The method of claim 42 or 43, wherein the weight loss is greater than the additive effect of administering the MTP inhibitor alone and the cholesterol absorption inhibitor alone.

45. The use of a combination of an MTP inhibitor and a cholesterol absorption inhibitor in the preparation of a medicament for the treatment of obesity.
